(19) United States
(12) Patent Application Publication
(10) Pub. No.: US 2024/0207325 A1
(43) Pub. Date: Jun. 27, 2024

(54) Pancreatic Islets as Protein Factories

(71) Applicant: BIOCRINE AB, Solna (SE)

(72) Inventors: Per-Olof Berggren, Solna (SE); Ingo Leibiger, Solna (SE); Barbara Leibiger, Solna (SE)

(21) Appl. No.: 18/554,136

(22) PCT Filed: Apr. 8, 2022
(86) PCT No.: PCT/EP2022/059516

§ 371 Date: Oct. 5, 2023

Related U.S. Application Data
(60) Provisional application No. 63/173,622, filed on Apr. 12, 2021.

(51) Int. Cl. A61K 35/39; A61K 38/22; A61K 38/26; A61P 3/04; C07K 14/575; C07K 14/605; C12N 5/071; C12N 15/86

(52) U.S. Cl. CPC A61K 35/39; A61K 38/2264; A61K 38/26; A61P 3/04; C07K 14/5759; C07K 14/605; C12N 5/0677; C12N 15/86; C12N 2533/50; C12N 2710/10043

(57) ABSTRACT

Engineered pancreatic islet organoids encoding a polypeptide not endogenously expressed in pancreatic islet cells and methods for using them to treat diseases and disorders associated with reduced or absent expression of the polypeptide are provided.

### CROSS REFERENCE

This application claims priority to U.S. Provisional Patent Application Ser. No. 63/173,622 filed Apr. 12, 2021, incorporated by reference herein in its entirety. Sequence Listing Statement:

A computer readable form of the Sequence Listing is filed with this application by electronic submission and is incorporated into this application by reference in its entirety. The Sequence Listing is contained in the file created on Apr. 5, 2022 having the file name “20-1719-WO-SeqList_ST25.txt” and is 3 kb in size.

### BACKGROUND

Pancreatic islets of Langerhans are micro-organs that form the endocrine part of the pancreas. They consist of endocrine alpha-, beta-, delta-, epsilon- and PP-cells, that produce and secrete glucagon, insulin, somatostatin, ghrelin, and pancreatic polypeptide, respectively. Islet organoids (also called pseudo-islets) that are formed by self-reassembly of islet cells following disaggregation and genetic manipulation, behave similarly to native islets.

### SUMMARY

In one aspect, the disclosure provides engineered pancreatic islet organoids comprising a recombinant expression vector, wherein the recombinant expression vector comprises (a) a nucleic acid sequence encoding a polypeptide, and (b) a suitable control sequence, operatively linked to the nucleic acid sequence. In one embodiment, the recombinant expression vector is present in pancreatic islet organoid cells that do not endogenously express the polypeptide. In another embodiment, the polypeptide is not endogenously expressed in pancreatic islet cells. In a further embodiment, the polypeptide is a therapeutic polypeptide, or analogues thereof. In some embodiments, the therapeutic polypeptide is selected from the group consisting of polypeptide hormones and enzyme replacement proteins. In other embodiments, the therapeutic polypeptide comprises a polypeptide hormone, selected from the group consisting of thyroid-stimulating hormone (TSH), prolactin, parathyroid hormone (PTH), leptin, follicle-stimulating hormone (FSH), and growth hormone (GH), or analogues thereof. In some embodiments, the therapeutic polypeptide comprises a polypeptide hormone selected from the group consisting of thyrotropin-releasing hormone (TRH), renin, gastrin, vasoactive intestinal peptide (VIP), vasopressin (ADH), oxytocin (OXY), melanocyte-stimulating hormone (MSH), calcitonin, cholecystokinin (CCK), atrial natriuretic peptide (ANP), angiotensin, amylin, and adrenocorticotropic hormone (ACTH), or analogues thereof. In other embodiments, the therapeutic polypeptide comprises an enzyme replacement protein, wherein the enzyme replacement protein is selected from the group consisting of factor VII (eptacog alfa), factor VIII, factor IX, factor XIII (catridecacog), Von Willenbrand factor, taliglucerase alfa, agalsidase alfa or beta, imiglucerase, velaglucerase alfa, alglucosidase alfa, galsulfase, domase alfa, laronidase, conestat alfa (C1 esterase inhibitor), pegloticase alpha-1-proteinase inhibitor, asfotase alfa (Strensiq), idursulfase, elosulfase alfa valiase, selbelipase alfa, epoetin teta (Eporatio), beta (NeoRecormon) zeta (Retacrit), darbepoetin alfa (Aranesp), luspatercept (Reblozyl), filgrastim, lenograstim, and Von Willebrand factor-cleaving protease, or analogues thereof. In various embodiments, the therapeutic polypeptide comprises a neurotrophic factor selected from the group consisting of enkephalin, endorphin, substance P, neurotensin, neuropeptide-Y, bombesin, brain-derived neutrophic factor (BDNF), nerve growth factor (NGF), neuotrophin-3, neurotrophin-4, ciliary neurotrophic factor (CNTF), leukemia inhibitory factor (LIF), cholinergic differentiation factor, cardiotrophin-1, oncostatin M, tumor necrosis factor (TNF), Neu differentiation factor, heregulin, acetylcholine receptor-inducing activity, glial growth factors (GGFs), glial cell line derived neurotrophic factor (GDNF), artemin, neurturin, persephin, osteogenic protein-1 (OP-1), bone morphogenetic proteins (BMPs), growth differentiation factors, ephrin, epidermal growth factor (EGF), insulin-like growth factors (IGF), platelet-derived growth factor (PDGF), hepatocyte growth factor (HGF), granulocyte-colony stimulating factor (G-CSF), serine protease inhibitors, protease nexin-1, hedgehog family of inducing proteins, agrin, laminin 2, neuroimmunophilins, pigment epithelium-derived factor (PEDF), activity-dependent neuroprotective protein (ADNP), neuritin (activity-induced neurotrophic factor), angiogenesis growth factor, cerebral dopamine neurotrophic factor (CDNF), mesencephalic astrocyte-derived neurotrophic factor (MANF), Peptide-6, davunetide (derived from ADNP), and cerebrolysin.

In some embodiments, the recombinant expression vector comprises a viral vector, including but not limited to a retroviral vector, a lentivirus vector, a murine leukemia virus (MMLV) vector, a murine stem cell virus (MSCV) vector, an adenoviral vector, a herpes simplex virus vector, a Baculovirus vector, and an adeno-associated viral vector. In other embodiments, the expression vector comprises a non-viral vector.

In some embodiments, the suitable control sequence is pancreatic tissue specific and is selected from the group consisting of a midkine (MK) promoter, a cyclooxygenase-2 (Cox2) M promoter, a Cox 2L promoter, a vascular endothelial growth factor (VEGF) promoter, a caveolin 1 promoter, a fms-like receptor tyrosine kinase 1 (FLT-1) promoter, a sloppy paired1 (SLP-1) promoter, a gastrin-releasing peptide (GRP) promoter, an epithelial glycoprotein 2 (EGP-2) promoter, an insulin promoter, and a glucagon promoter. In other embodiments, the recombinant expression vector comprises an adeno associated viral vector and the insulin promoter is RIP1 or RIP2. In further embodiments, the suitable control sequence is selected from the group consisting of a cytomegalovirus (CMV) promoter, a chicken β-actin (CAG) promoter, a Rous sarcoma virus (RSV), a simian virus 40 (SV40), a mammalian elongation factor 1α (EF1α) promoter, a muscle creatine kinase (MCK) promoter, a human phosphoglycerate kinase 1 (PGK1) promoter, and a tetracycline-responsive element (TRE)-tight promoter.

In another embodiment, the disclosure provides compositions comprising the engineered pancreatic islet organoid of any embodiment or combination of embodiments herein and a silk matrix. In one embodiment, the silk is spider silk. In another embodiment, the silk is functionalized with a cell-binding-motif.

In another aspect, the disclosure provides uses and methods for treating a disorder comprising administering the engineered pancreatic islet organoid of any embodiment or combination of embodiments herein, or compositions thereof, to treat a disorder. In one embodiment, the engineered pancreatic islet organoid or composition is implanted in in the eye, pituitary, pancreas, small intestine, stomach, brain, kidney, parathyroid gland, duodenum, thyroid, liver, heart, ovary, testicles, fat, and/or skin of the subject in need thereof. In another embodiment, the nucleic acid sequence encodes a polypeptide listed in the left hand column of Table 1, and the subject has a disorder listed in the right hand column of Table 1 in the same row as the polypeptide.

In another aspect, the disclosure provides a process for producing an engineered pancreatic islet organoid containing islet cells, comprising the steps of (a) introducing a recombinant expression vector into pancreatic islet cells, wherein the recombinant expression vector comprises (i) a nucleic acid sequence encoding a polypeptide not endogenously expressed in pancreatic islet cells, and (ii) a suitable control sequence operatively linked to the nucleic acid sequence; and (b) culturing the pancreatic islet cells containing the recombinant expression vector in vitro to form the engineered pancreatic islet organoid containing islet cells. In one embodiment, the introducing step (a) further comprising the steps (a1) preparing an aqueous mixture of the pancreatic islet cells containing the recombinant expression vector with a silk protein capable of assembling into a water-insoluble macrostructure, optionally further containing laminins; (a2) allowing the silk protein to assemble into a water-insoluble macrostructure in the presence of the pancreatic islet cells containing the recombinant expression vector, thereby forming a 3D silk matrix for the pancreatic islet cells containing the recombinant expression vector; and wherein the culturing step (b) involves culturing the pancreatic islet cells containing the recombinant expression vector in vitro within the silk matrix to form the engineered pancreatic islet organoid containing islet cells. In another embodiment, the process further comprises (c) placing the engineered pancreatic islet organoid containing islet cells on a 3D silk matrix consisting of a water-insoluble macrostructure of a silk protein, optionally further containing laminins; and (d) allowing the engineered pancreatic islet organoid containing islet cells to adhere to the 3D silk matrix.

### DESCRIPTION OF DRAWINGS

FIG. 1a-c shows in vitro characterization of leptin-expressing islet organoids. (a) Schematic illustration of vAd-RIP-leptin-OFF. The rat insulin-1 promoter (rIns-1) drives expression of the synthetic transcription factor rTA (Tet-off) and the green fluorescent protein ZsGreen™ in pancreatic beta-cells. The TRE-tight promoter drives expression of mouse leptin and the red fluorescent protein mCherry™. The two expression cassettes are separated by a transcription blocker sequence (TB). Binding of rTA to the TRE-tight promoter induces in the absence of doxycycline the expression of leptin and mCherry™, while addition of doxycycline turns-off the expression of the two proteins. IRES-elements between the elements ensures stoichiometric expression of the two proteins under the same promoter. (b) Representative maximum projection of a confocal imaging 3D-stack of leptin-expressing islet organoids cultured for 4 weeks in vitro (1^{st} set of experiments), showing reflection, expression of ZsGreen™, mCherry™ and their overlay. Scale bar: 50 μm. (c) Representative maximum projection of an confocal imaging 3D-stack of leptin-expressing islet organoids cultured for 4 weeks in vitro (2^{nd} set of experiments), showing reflection, expression of ZsGreen™, mCherry™ and their overlay. Scale bar: 50 μm.

FIG. 2a-g shows in vivo characterization of leptin-expressing islet organoids from the first set of experiments. (a) Photograph of the eye containing the leptin-expressing islet organoid graft (red arrow) 9 weeks after transplantation. (b) Maximum projection of a 3D-stack of a leptin-expressing islet organoid graft (red arrow) obtained by confocal imaging 9 weeks after transplantation; (ba) reflection, (bb) mCherry™ fluorescence, (bc) ZsGreen™ fluorescence, (bd) overlay image; scale bar 500 μm. (c) Bodyweight of transplanted and control mice over the period of the experiment. (d) Area under the curve (AUC) for ipGTT of transplanted and control mice over the period of the experiment. (e) Fasting blood glucose of transplanted and control mice over the period of the experiment. (f) Plasma insulin levels of transplanted and control mice at the end of the experiment. (g) Plasma C-peptide levels of transplanted and control mice at the end of the experiment. (d, e) *p<0.05. (c-g) n=3.

FIG. 3a-g shows in vivo characterization of leptin-expressing islet organoids from the second set of experiment. (a) Photograph of the eye containing the leptin-expressing islet organoid graft (red arrow) 6 weeks after transplantation. (b) Maximum projection of a 3D-stack of a leptin-expressing islet organoid graft (red arrow) obtained by confocal imaging 12 weeks after transplantation; (ba) reflection, (bb) mCherry™ fluorescence, (bc) ZsGreen™ fluorescence, (bd) overlay image; scale bar 300 μm. (c) Bodyweight, (d) Area under the curve (AUC) for ipGTT, (e) Fasting blood glucose, (f) Plasma insulin levels and (g) Plasma C-peptide levels of transplanted and control mice over the period of the experiment. (c-g) *p<0.05, **p<0.01, ***p<0.001; n=7 for transplanted and 5 for control group.

FIG. 4a-d shows characterization of leptin-expressing islet organoid grafts from animals treated with doxycycline, removed at the end of the experiment. (a) Fluorescent microscopy image of a leptin-expressing islet organoid graft from transplanted non-doxycycline-treated animal; (aa) DAPI staining; (ab) ZsGreen™ fluorescence; (ac) mCherry™ fluorescence; (ad) overlay. (b) Fluorescent microscopy image of a leptin-expressing islet organoid graft from transplanted doxycycline-treated animal; (ba) DAPI staining, (bb) ZsGreen™ fluorescence, (bc) mCherry™ fluorescence (bd) overlay. (c) Immunohistochemistry image of a leptin-expressing islet organoid graft from transplanted non-doxycycline treated animal; (ca) DAPI staining, (cb) C-peptide staining, (cc) leptin staining, (cd) overlay. (d) Immunohistochemistry image of a leptin-expressing islet organoid graft from transplanted doxycycline treated animal; (da) DAPI staining, (db)C-peptide staining, (dc) leptin staining, (dd) overlay. (a-d) All scale bars 50 μm.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

All embodiments of any aspect of the disclosure can be used in combination, unless the context clearly dictates otherwise.

In a first aspect, the disclosure provides an engineered pancreatic islet organoid comprising a recombinant expression vector, wherein the recombinant expression vector comprises (a) a nucleic acid sequence encoding a polypeptide, and (b) a suitable control sequence operatively linked to the nucleic acid sequence.

The instant disclosure provides the novel and surprising findings that pancreatic islet organoids can be engineered to express any polypeptides, including non-pancreatic polypeptides (defined as polypeptides not normally produced in pancreatic islets), and that these engineered pancreatic islet organoids can be used as protein factories, implantable in subjects, for long term treatment of diseases that are treatable using the polypeptides produced by the engineered pancreatic islet organoids. The engineered pancreatic islet organoids mimic a miniature organ. The engineered pancreatic islet organoids have a structure and functionality that make it well suited to excrete polypeptides into the bloodstream of a subject, and this functionality can be maintained over long periods of time as compared to surgical implantation of individual cell-types. The islet organoid can be implanted within or adjacent to other organs, in effect becoming a ‘complete organ within another organ’. This is a unique feature of engineered pancreatic islet organoids.

The therapeutic effect of the polypeptides can be further tailored for an enhanced local effect, by choosing a particular transplantation site of the implant, depending on the disease. The compositions and methods disclosed herein are exemplified, in a non-limiting fashion, by the creation of pancreatic islet organoids engineered to express leptin. Leptin is a polypeptide not normally produced by pancreatic islets. The leptin producing engineered pancreatic islet organoids were then successfully used to rescue the metabolic phenotype in ob/ob (leptin deficient knock-out) mice, as shown in the Examples.

As used herein, an “engineered pancreatic islet organoid” is an in vitro generated cell cluster that mimics structure and function (insulin glucagon, amylin, somatostatin, ghrelin and pancreatic polypeptide secretion) of a pancreatic islet of Langerhans, wherein the cell cluster comprises at least pancreatic beta cells, but can include pancreatic alpha-, Pancreatic Polypeptide (PP)-,epsilon- and delta-cells. In various embodiments, the engineered pancreatic islet organoids comprises at least beta cells and alpha cells; in other embodiments, engineered pancreatic islet organoids comprise at least pancreatic beta cells, alpha cells, and one, two, or all three of PP-cells, epsilon-cells and delta-cells.

Pancreatic islets of Langerhans are multi-cellular, micro-organs that form the endocrine part of the pancreas. Islets comprise fenestrated blood vessels, which allow for an efficient exchange of blood-derived factors, which can stimulate the islet and result in the release of the polypeptide hormones produced by the islets into general blood circulation.

In one embodiment, the pancreatic islet organoid cells can be engineered from islets that are obtained from any animal that has pancreatic islets of Langerhans suitable for use, including but not limited to any mammal such as a human. In other embodiments, the engineered pancreatic islet organoid can be generated from induced pluripotent stem cell or stem cell from any source using any suitable techniques, including but not limited to those disclosed in US 20190211310, incorporated by reference in its entirety. In one non-limiting embodiment, the engineered pancreatic islet organoid can be generated by culturing an induced pluripotent stem cell (iPSC)-derived or human embryonic-derived or cadaveric or human-derived) beta-like cell, and optionally, a structural component, in a 3-dimensional matrix. In various embodiments, the pancreatic islet organoid can be generated by culturing the iPSC-derived (or human embryonic-derived or cadaveric or human-derived) beta-like cell with an adipose-derived stem cell and/or an endothelial cell. In various embodiments, the pancreatic islet organoid can be generated by culturing the iPSC-derived (or human embryonic-derived or cadaveric or human-derived) beta-like cell with an iPSC-derived (or human embryonic-derived or cadaveric or human-derived) alpha-like cell, an iPSC-derived (or human embryonic-derived or cadaveric or human-derived) delta-like cell, an iPSC-derived (or human embryonic-derived or cadaveric or human-derived) epsilon-cells, iPSC-derived (or human embryonic-derived or cadaveric or human-derived) PP-cells, and/or an iPSC-derived (or human embryonic-derived or cadaveric or human-derived) duct-like cell.

In various non-limiting embodiments, the engineered pancreatic islet organoids of the disclosure can contain any one or more of the following cell types: iPSC-derived (or human embryonic-derived or cadaveric or human-derived) beta-like cells, iPSC-derived (or human embryonic-derived) alpha-like cells, iPSC derived (or human embryonic-derived or cadaveric or human-derived) delta-like cells, iPSC-derived (or human embryonic-derived or cadaveric or human-derived) PP cells, iPSC-derived (or human embryonic-derived or cadaveric or human-derived) epsilon cells, and iPSC-derived (or human embryonic-derived or cadaveric or human-derived) duct-like cells. In some embodiments, the iPSCs are human iPSCs (hiPSC). In some embodiments, the stem cells comprise human embryonic stem cells (hESC). In some embodiments, the pancreatic islet organoid comprises adipose-derived stem cells and/or endothelial cells.

Any of the cells in the engineered pancreatic islet organoid can be engineered to contain the recombinant expression vector, including but not limited to, alpha-cell, beta-cell, delta-cells, PP cells, epsilon cells or duct-like cells. In one embodiment, the beta cells comprise the recombinant expression vector and express a polypeptide not endogenously expressed by beta cell and/or a non-pancreatic polypeptide.

The recombinant expression vector can be any vector suitable for expression in pancreatic islet organoids, including viral and non-viral vectors. Suitable viral vectors include, but are not limited to, retroviral vectors, lentivirus vector, murine leukemia virus (MMLV) vector, murine stem cell virus (MSCV) vector, adenoviral vector, herpes simplex virus vector, Baculovirus vector, and adeno-associated viral vectors. In one, non-limiting example, the recombinant expression vector is an adenoviral vector.

Any suitable nucleic acid sequence encoding a polypeptide for expression in the islets may be used. As used herein, the term “nucleic acid sequence” refers to DNA molecules (e.g., recombinant DNA, cDNA, genomic DNA, plastid DNA, mitochondrial DNA) and RNA molecules (e.g., mRNA) and analogs of the DNA or RNA generated using nucleotide analogs.

In some embodiments, the nucleic acid sequence encoding a polypeptide may be modified from known coding sequences by, for example, changes in the nucleic acid sequence due to the degeneracy of the genetic code; codon optimization of the nucleic acid sequence for expression in pancreatic islet organoids; changes in the nucleic acid sequence to introduce at least one amino acid substitution, insertion, deletion and/or addition; removal of one or more intron; insertion of one or more heterologous introns; deletion of one or more upstream or downstream regulatory regions; insertion of one or more heterologous upstream or down-stream regulatory regions; deletion of the 5′ and/or 3′ untranslated region; insertion of a heterologous 5′ and/or 3′ untranslated region; and modification of a polyadenylation site.

As used throughout the present application, the term “polypeptide” is used in its broadest sense to refer to a sequence of subunit amino acids of any length. The polypeptides of the invention may comprise L-amino acids, D-amino acids (which are resistant to L-amino acid-specific proteases in vivo), or a combination of D- and L-amino acids.

The recombinant expression vector comprises a suitable control sequence that is operatively linked to the nucleic acid sequence encoding the polypeptide. The control sequence can be any control sequence capable of effecting expression of the nucleic acid sequence encoding the polypeptide. The control sequence can be either pancreatic-tissue specific, islet cell specific (i.e.: beta cell specific; alpha cell specific; delta cell specific; etc.) or non-pancreatic-tissue specific (i.e., a general control sequence). Non-limiting examples of pancreatic tissue specific control sequences include midkine (MK) promoter, Cox2M promoter, Cox 2L promoter, vascular endothelial growth factor (VEGF) promoter, caveolin 1 promoter, fms-like receptor tyrosine kinase 1(FLT-1) promoter, sloppy paired-1 (SLP-1) promoter, gastrin-releasing peptide (GRP) promoter, epithelial glycoprotein 2 (EGP-2) promoter, rat insulin promoter (RIP1 or RIP2) (beta cell specific), insulin promoters (beta cell specific), glucagon promoter (alpha cell specific), and somatostatin promoters (delta cell specific). Non-limiting examples of non-tissue specific control sequences include cytomegalovirus (CMV) promoter, chicken R-actin (CAG) promoter, Rous sarcoma virus (RSV), simian virus 40 (SV40), mammalian elongation factor 1α (EF1α) promoter, muscle creatine kinase (MCK) promoter, human phosphoglycerate kinase 1 (PGK1) promoter, and tetracycline-responsive element (TRE)-tight promoter. In one embodiment, the promoter can be an inducible promoter such as a Tet-ON/OFF promoter, a pLac promoter, or a pBad promoter. In one, non-limiting example, the control sequence comprises pTRE-tight promoter. In one embodiment, the promoter can be a synthetic promoter comprising transcriptional control elements of the above-mentioned promoters.

The expression vector of the instant disclosure can include any other components appropriate for use with the vector. The control sequences need not be contiguous with the nucleic acid sequences, as long as they function to direct the expression thereof. Thus, for example, intervening untranslated yet transcribed sequences can be present between a promoter sequence and the nucleic acid sequences and the promoter sequence can still be considered “operatively linked” to the nucleic acid. The expression vector may comprise other control sequences including, but not limited to, polyadenylation signals, termination signals, and ribosome binding sites. The control sequence used to drive expression of the disclosed nucleic acid sequences may be constitutive or inducible (driven by any of a number of inducible promoters including, but not limited to, tetracycline, ecdysone, steroid-responsive). In one embodiment, he expression vector in the pancreatic islet organoids is replicable by integration into host chromosomal DNA.

In a further embodiment, the nucleic acid sequence encoding a polypeptide, or an expression promoter, may be introduced into the islet cells using the CRISPR/Cas9 system, other nucleases such as zinc-finger nucleases or transcription activator-like effector nucleases (TALEN), or other suitable gene editing system.

In one embodiment of the first aspect, the recombinant expression vector of the engineered pancreatic islet organoid is present in pancreatic islet organoid cells that do not endogenously express the polypeptide. In this embodiment, the recombinant expression vector is only present in the pancreatic cell type(s), which do not endogenously express the polypeptide; this allows the cells containing the recombinant expression vector to produce a polypeptide that the cell would not normally produce.

In one non-limiting example, the engineered pancreatic islet organoid comprise beta cells containing a recombinant expression vector, which comprises a nucleic acid encoding a polypeptide that is not endogenously expressed by pancreatic beta cells, but may be endogenously expressed by other pancreatic islet cells. In a further example, the engineered pancreatic islet organoid comprises alpha cells containing a recombinant expression vector, which comprises a nucleic acid encoding a polypeptide that is not endogenously expressed by alpha cells, but may be endogenously expressed by other pancreatic islet cells. In a further example, the engineered pancreatic islet organoid comprises delta cells containing a recombinant expression vector, which comprises a nucleic acid encoding a polypeptide that is not endogenously expressed by delta cells, but may be endogenously expressed by other pancreatic islet cells. Examples of pancreatic polypeptides, which are differentially expressed in different pancreatic cell types, include, but are not limited to glucagon, insulin, amylin, glucagon-like peptide-1 (GLP-1), somatostatin, and vasoactive intestinal peptide (VIP). This embodiment permits, for example, the engineered pancreatic islet organoid to produce higher concentrations of a polypeptide, which is normally produced in pancreatic islet cells at a very low concentration, and/or may permit expression in an islet cell type that permits secretion of the polypeptide under certain conditions. In one example, the engineered pancreatic islet organoid can be engineered to express VIP, which is chiefly produced in the gut, but can be produced in low concentrations in pancreatic islet cells. Thus, in this embodiment, the engineered pancreatic islet organoids expressing VIP can be used to increase the concentration of VIP expressed by the pancreatic islet cells. In another non-limiting embodiment, a recombinant expression vector capable of expressing GLP-1 is present in pancreatic islet beta cells of the engineered pancreatic islet organoid; expression and secretion of GLP-1 permits rapid local delivery of GLP-1 to the beta cells in response to high glucose conditions, thus providing for improved diabetes therapy. In various examples of the embodiment, the recombinant expression vector comprises an adenovirus or adeno-associated viral vector and the suitable control sequence comprises the CMV promoter or an insulin promoter including but not limited to a human insulin promoter of functional portion thereof that can direct gene expression, and rat insulin promoter such as RIP1 and RIP2.

In non-limiting examples, the encoded polypeptides can include synthetic (non-naturally occurring) polypeptides, polypeptide fragments, non-human polypeptides and natural or synthetic polypeptide analogs.

In a further embodiment of the first aspect, the polypeptide expressed by the engineered pancreatic islet organoid is not endogenously expressed in any pancreatic islet cells types. In this embodiment, the encoded polypeptide is not endogenously expressed by non-engineered pancreatic islet cells.

In all of these embodiments, the polypeptide may be a therapeutic polypeptide. Examples of such therapeutic polypeptides include, but are not limited to, polypeptide hormones or enzymatic replacement proteins.

In one embodiment, the therapeutic polypeptides comprise polypeptide hormones that are not endogenously expressed in the pancreatic islet. Non-limiting examples of polypeptide hormones that are not endogenously expressed in the pancreatic islet include parathyroid hormone (PTH), leptin, follicle-stimulating hormone (FSH), Growth Hormone (GH), thyrotropin-releasing hormone (TRH), renin, gastrin, vasopressin (ADH), oxytocin (OXY), melanocyte-stimulating hormone (MSH), calcitonin, cholecystokinin (CCK), atrial natriuretic peptide (ANP), angiotensin, leutinizing hormone releasing hormone (LHRH), and adrenocorticotropic hormone (ACTH). In embodiments where the polypeptide hormone is processed after expression, the nucleic acid may express the mature form or the pre(pro)-protein version of the polypeptide hormone. As used herein, the mature form of a polypeptide hormone is the form of the polypeptide hormones after final processing. In one embodiment, the nucleic acid encodes the mature form of a polypeptide hormone.

In another embodiment, the therapeutic polypeptides comprise incretins or enzyme replacement proteins. The nucleic acid may encode any such incretin, including but not limited to, glucagon-like peptide-1 (GLP-1), oxyntomodulin (OXM), glucose-dependent insulinotrophic peptide (GIP), GLP-1 Receptor binding polypeptide, GIP Receptor binding polypeptide, Glucagon Receptor binding polypeptide, and dual or triple combined Glucagon, GIP, glucagon Receptor binding polypeptide(s). The nucleic acid may encode any such enzymatic replacement protein, including, but not limited to factor VII (eptacog alfa), factor VIII, factor IX, factor XIII (catridecacog), Von Willenbrand factor, taliglucerase alfa, agalsidase alfa or beta, imiglucerase, velaglucerase alfa, alglucosidase alfa, galsulfase, dornase alfa, laronidase, conestat alfa (C1 esterase inhibitor), pegloticase alpha-1-proteinase inhibitor, asfotase alfa (Strensiq), idursulfase, elosulfase alfa valiase, selbelipase alfa, epoetin teta (Eporatio), beta (NeoRecormon) zeta (Retacrit), darbepoetin alfa (Aranesp), luspatercept (Reblozyl), filgrastim, lenograstim, and Von Willebrand factor-cleaving protease.

In a further embodiment, the therapeutic polypeptides can further comprise neuropeptides and neurotrophic factors that are not endogenously expressed in the pancreatic islet. The nucleic acid may encode any such neuropeptide or neurotrophic factor, including, but not limited to enkephalin, endorphin, substance P, neurotensin, neuropeptide-Y, bombesin, brain-derived neutrophic factor (BDNF), nerve growth factor (NGF), neuotrophin-3, neurotrophin-4, ciliary neurotrophic factor (CNTF), leukemia inhibitory factor (LIF), cholinergic differentiation factor, cardiotrophin-1, oncostatin M, growth promoter activity factor, tumor necrosis factor (TNF), Neu differentiation factor, heregulin, acetylcholine receptor-inducing activity, glial growth factors (GGFs), glial cell line derived neurotrophic factor (GDNF), artemin, neurturin, persephin, osteogenic protein-1 (OP-1), bone morphogenetic proteins (BMPs), growth differentiation factors, ephrin, epidermal growth factor (EGF), transforming growth factor (TGFα and TGFβ), insulin-like growth factors (IGF), platelet-derived growth factor (PDGF), hepatocyte growth factor (HGF), granulocyte-colony stimulating factor (G-CSF), serine protease inhibitors, protease nexin-1, hedgehog family of inducing proteins, agrin, laminin 2, ACh-inducing activity (ARIA), neuroimmunophilins, pigment epithelium-derived factor (PEDF), activity-dependent neuroprotective protein (ADNP), neuritin (activity-induced neurotrophic factor), angiogenesis growth factor, vascular endothelial growth factor (VEGF), cerebral dopamine neurotrophic factor (CDNF), mesencephalic astrocyte-derived neurotrophic factor (MANF), Peptide-6, davunetide (derived from ADNP), and cerebrolysin.

It will be understood that the engineered pancreatic islet organoid may include multiple recombinant expression vectors capable of expressing the same or different polypeptides. In various embodiments, the engineered pancreatic islet organoids may comprise 2, 3, or more different recombinant expression vectors, each capable of expressing the same or a different polypeptide. The different recombinant expression vectors may all be present in the same cell type(s) in the organoid, or may be present in different cell type(s) in the organoid. In one non-limiting embodiment, the organoid may comprise 2 or more different expression vectors that all express the same polypeptide but are each under the control of a different promoter or other suitable control element such that the different expression vectors express the polypeptide under different conditions and/or only when present in an organoid cell type in which the promoter and/or control element is active. In another non-limiting embodiment, the organoid may comprise 2 or more different expression vectors that all express a different polypeptide, where each different polypeptide may be under the control of the same or a different promoter or other suitable control element such that the different expression vectors may express the different polypeptide under the same conditions (when same promoter and/or control elements is used), under different conditions (when different promoters and/or control elements are used), and/or only when present in an organoid cell type in which the promoter and/or control element is active. In one specific embodiment, the organoids may comprise 2 or 3 of the following: (1) a first expression vector, wherein expression is under control of the insulin promoter to limit expression to beta cells; (2) a second expression vector, wherein expression is under control of the glucagon promoter to limit expression to alpha cells; and (3) a third expression vector, wherein expression is under control of the somatostatin promoter to limit expression to delta cells. In other examples, the organoid may comprise one or more expression vector wherein expression is under the control of a CMV promoter and/or the rat insulin promoter, RIP1 or RIP2.

In a second aspect, the disclosure provides a composition comprising the engineered pancreatic islet organoid of any embodiment or combination of embodiments disclosed herein and a silk matrix. The engineered pancreatic islet organoid may be adhered on top of the silk matrix or integrated within the silk matrix. The silk matrix may have the form of a fiber, foam, film, fiber mesh, capsule, net, or gel, preferably a fiber or foam. The silk matrix can comprise a silk protein. The silk protein is preferably a fibroin, such as a silkworm fibroin, or a spider silk protein.

Spider silk is a biocompatible material, which can be made through recombinant DNA technology. Spider silk can serve as a cell scaffold material for the cultivation of eukaryotic cells. A polymer of silk protein can be used by the cells as a cell-scaffold. Spiders have up to seven different glands, which produce a variety of silk types with different mechanical properties and functions, including but not limited to dragline silk.

Spider silk proteins can form macrostructures, including films and foams, with attached and/or integrated cells, such as those of the islet organoid, and thus provide internal 3D support for the engineered pancreatic islet organoid cells. These microstructures provide a high seeding efficiency, yielding quickly and viably adhered pancreatic islet organoids. Compared to cultivation in other cell scaffolds, cells in spider silk attain a more tissue-like spreading when integrated into silk scaffolds. This may improve functionality and viability of the transplanted islet organoids.

Functionalized silk are bioactive silk-based materials with enhanced functionality for applications related to medicine and biotechnology. As used herein, “functionalized spider silk” means recombinant spider silk modified to include cell-binding motifs and covalently bound functional groups or domains. Examples of functionalized spider silk and methods of producing and using it, include but are not limited to those disclosed in WO2007078239A2, US20160024464, U.S. Ser. No. 10/316,069, and US20190144819, incorporated by reference in their entirety. In certain preferred embodiments of these and other aspects, the silk protein contains a cell-binding motif, such as a cell-binding motif selected from RGD, IKVAV (SEQ ID NO: 1), YIGSR (SEQ ID NO: 2), EPDIM (SEQ ID NO: 3), NKDIL (SEQ ID NO: 4), GRKRK (SEQ ID NO: 5), KYGAASIKVAVSADR (SEQ ID NO: 6), NGEPRGDTYRAY (SEQ ID NO: 7), PQVTRGDVF™ (SEQ ID NO: 8), AVTGRGDSPASS (SEQ ID NO: 9), TGRGDSPA (SEQ ID NO: 10), CTGRGDSPAC (SEQ ID NO: 11) and FN_{cc}; and preferably from FN_{cc}, GRKRK, IKVAV, RGD and CTGRGDSPAC, more preferably FN_{cc} and CTGRGDSPAC; wherein FN_{cc} is C¹X²X²RGDX³X⁴X⁵C²; wherein each of X¹, X², X³, X⁴ and X⁵ are independently selected from natural amino acid residues other than cysteine; and C¹ and C² are connected via a disulfide bond. A preferred group of cell-binding motifs are FN_{cc}, GRKRK, IKVAV, and RGD, and in particular FN_{cc}, such as CTGRGDSPAC.

Functionalized spider silks may comprise additional functional properties, for example, electrical conductance, cell-binding ability, growth factor bioactivity, molecular affinity, antimicrobial properties and enzymatic activity. Such additional functional properties may be added, for example, by dip-coating, genetic engineering, and/or enzymatic coupling of the functionalized spider silk. (Example of specifics of silk protein from patent WO2017137611A1.) In some embodiments, the functionalized spider silk can include immune suppressive proteins or other immune modulators in order to protect the engineered pancreatic organoids from an immune response, such as PD-L1. The functionalized spider silk can also include factors, which increase vascularization or other growth factors, such as Fibronectin, VEGF, FGF and GLP-1.

A composition comprising the engineered pancreatic islet organoid and a silk matrix can be prepared in various ways. The engineered pancreatic islet organoid can first be prepared separately, and then be placed on top of a silk matrix and allowed to adhere. Alternatively, the engineered pancreatic islet organoid can first be prepared separately, and then be placed in a silk solution, which is allowed to form a silk matrix encompassing the engineered pancreatic islet organoid. In a further preferred embodiment, a method for providing the composition comprising the engineered pancreatic islet organoid and a silk matrix comprises: (i) providing an aqueous solution of a silk protein capable of assembling into a water-insoluble macrostructure; (ii) preparing an aqueous mixture of a sample of the engineered islet cells with the silk protein, optionally further containing laminins; (iii) allowing the silk protein to assemble into a water-insoluble macrostructure in the presence of the engineered islet cells, thereby forming a 3D silk matrix for the eukaryotic cells; and (iv) maintaining and differentiating the engineered islet cells within the silk matrix under conditions suitable to obtain an engineered pancreatic islet organoid. In a further embodiment, the methods can comprise that the engineered pancreatic islet organoid can be detached from the silk matrix and be grown in suspension as desired.

In a third aspect, the disclosure provides uses of the engineered pancreatic islet organoids of the first aspect of the disclosure or the compositions of the second aspect of the disclosure to treat a disorder. In a fourth aspect, the disclosure provides methods for treating a disorder, comprises implanting into a subject having a disorder the engineered pancreatic islet organoid or composition of any embodiment or combination of embodiments of the disclosure, in an amount effective to treat the disorder. As discussed above, the instant disclosure provides the novel and surprising findings that pancreatic islet organoids can be engineered to express any polypeptide, including non-pancreatic polypeptides, and that these engineered pancreatic islet organoids can be used to treat diseases that are treatable using the any polypeptide, including non-pancreatic polypeptides produced by the engineered pancreatic islet organoids.

As used herein, the term “subject” is any subject for which treatment is desired, such as mammalian subjects including but not limited to humans, cattle, dogs, cats, guinea pigs, rabbits, rats, mice, cattle, horses, and so on. In one embodiment, the subject is human. As defined herein “a subject in need thereof” is a subject suffering from a disorder associated with a reduced expression level, or the absences of expression, of a polypeptide not endogenously expressed by pancreatic islets.

In one embodiment of the uses and methods of the disclosure, the engineered pancreatic islet organoid or composition may be implanted in a subject. The implantation can be accomplished through any suitable procedure and the engineered pancreatic islet organoid can be implanted to any suitable area of the subject, including but not limited to, the eye, pituitary, pancreas, small intestine, stomach, brain, kidney, parathyroid gland, duodenum, thyroid, liver, heart, ovary, testicles, fat, or skin of the subject. In one embodiment, the engineered pancreatic islet organoid is implanted into an eye of the subject.

The uses and methods can be used to treat any disorder associated with or caused by reduced levels of, or the complete absence of, the expression of a circulating polypeptide. In various embodiments, the disorder to be treated and the polypeptide expressed by the engineered pancreatic islet organoid of the disclosure for treating the disorder are listed in the same row of Table 1 below. Thus, for example, when the engineered pancreatic islet organoids express thyroid-stimulating hormone, the uses and methods may comprise treating a subject with hypothyroidism (see Row 2 of Table 1). Similarly, when the engineered pancreatic islet organoids express prolactin, the uses and methods may comprise treating a subject with hypoprolactinemia (see Row 3 of Table 1). It will thus be clear to those of skill in the art how to determine which disorder may be treated by engineered pancreatic islet organoids expressing which polypeptide by viewing each row of Table 1.

TABLE 1
Polypeptide and Associated Disease and Disorders

| Polypeptide | Associated Disorders |
|---|---|
| thyroid-stimulating hormone (TSH) | Hypothyroidism |
| Prolactin | hypoprolactinemia, |
| parathyroid hormone (PTH) | hypoparathyroidism, hypocalcemia |
| Leptin | obesity, hypertriglyceridemia, lipodystrophy |
| | (Berardinelli-Seip syndrome, Lawrence |
| | syndrome, and Barraquer-Simons syndrome) |
| Soluble Leptin Receptor (sLR) | obesity, hypertriglyceridemia, diabetes, |
| | metabolic syndrome |
| follicle-stimulating hormone (FSH) | polycystic ovarian syndrome, Kallmann's |
| | syndrome, hypothalamic suppression, |
| | hypopituitarism, hyperprolactinemia, |
| | gonadotropin deficiency |
| Growth Hormone (GH) | growth hormone deficiency, Turner |
| | syndrome, chronic kidney failure, Prader- |
| | Willi syndrome, intrauterine growth |
| | restriction, and idiopathic short stature |
| thyrotropin-releasing hormone (TRH) | hypothyroidism, depression |
| Gastrin | Hypochlorhydria |
| vasopressin (ADH) | diabetes insipidus |
| oxytocin (OXY) | induce labor, autism, Prader-Willi syndrome |
| melanocyte-stimulating hormone (MSH) | sleep disorders, erythropoietic protoporphyria, |
| | polymorphous light eruption, actinic |
| | keratosis, low libido |
| Calcitonin | bipolar disorder, osteoporosis, |
| | hypocalcaemia, Paget's disease, bone |
| | metastases, phantom limb pain, spinal |
| | stenosis |
| cholecystokinin (CCK) | digestive dysfunction and anxiety |
| atrial natriuretic peptide (ANP) | heart failure, kidney disease, heart disease |
| Angiotensin | hypotension, heart disease, kidney disease |
| amylin | Obesity, diabetes |
| vasoactive intestinal peptide (VIP) | osteoarthritis and VIPoma |
| adrenocorticotropic hormone (ACTH) | Addison's disease, Sheehan's syndrome, |
| | hypocortisolism, hypopituitarism, West |
| | syndrome, multiple sclerosis |
| Alglucerase, Imiglucerase, Velaglucerase | Gaucher's disease |
| alfa, Taliglucerase alfa | |
| Agalsidase alfa or beta | Fabry's disease |
| Alglucosidase alfa | Pompe's disease |
| Laronidase, idursulfase, elosulfase alfa, | Mucopolysaccharidosis |
| galsulfase | |
| Selbelipase alfa | Wolman disease |
| Conestat alfa | hereditary angioedema |
| Filgrastim, lenograstim | chronic neutropenia |
| Epoetin teta, epoetin beta, epoetin zeta, | renal anemia (dialysis and other subjects) |
| darbepoetin alfa | |
| Luspatercept | thalassemia beta |
| Factor VII, Factor VIII, Factor IX, Factor | hemophilia, von Willebrand disease, factor |
| XIII, von Willebrand factor | XIII deficiency |
| von Willebrand factor-cleaving protease | thrombotic thrombocytopenia purpura (TTP) |
| (ADAMTS13) | and hemolytic-uremic syndrome (HUS) |
| Valiase | Phenylketonuria |
| Alpha-1 proteinase inhibitor | pulmonary emphysema |
| IL-1 receptor antagonist (IL-1RA), Anakinra | rheumatoid Arthritis (RA), autoinflammatory |
| | disorders, systemic juvenile idiopathic |
| | arthritis, gout, cryopyrin-associated periodic |
| | syndromes (CAPS) |
| Asfotase alfa | Hypophosphatasia |
| Dornase alfa | cystic fibrosis |
| glucagon-like peptide-1 (GLP-1) | diabetes, obesity, metabolic syndrome |
| glucose-dependent insulinotrophic peptide | diabetes, obesity, metabolic syndrome |
| (GIP) | |
| Oxyntomodulin | diabetes, obesity, metabolic syndrome |
| GLP-1 Receptor binding polypeptide | diabetes, obesity, metabolic syndrome |
| GIP Receptor binding polypeptide | diabetes, obesity, metabolic syndrome |
| Glucagon Receptor binding polypeptide | diabetes, obesity, metabolic syndrome |
| dual or triple combined Glucagon, GIP, | diabetes, obesity, metabolic syndrome |
| glucagon Receptor binding polypeptide | |
| Encephalin | pain, depression, neurotransmission-related |
| | diseases |
| Endorphin | pain, psychiatric disease including depression, |
| | sedation, stress disorders |
| substance P | pain, depression, gastrointestinal disease, |
| | neurodegenerative disease |
| neurotensin | neurodegenerative disease including |
| | Alzheimer's and Parkinson's disease, |
| | schizophrenia |
| neuropeptide-Y | eating disorders, cardiovascular disease, |
| | seizure-related disease, stress-related disease |
| Bombesin | Obesity |
| brain-derived neutrophic factor (BDNF) | neurodegenerative and neuromuscular disease |
| | including Huntington's, Alzheimer's, |
| | Parkinson's disease, multiple sclerosis, motor |
| | neuron disease, amyotrophic lateral Sclerosis, |
| | ataxia, muscular dystrophy, spinal muscular |
| | atrophy, stroke, spinal cord injury, Charcot- |
| | Marie-Tooth disease, progressive |
| | supranuclear palsy |
| nerve growth factor (NGF) | neurofibroma, diabetic neuropathy, |
| | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| neuotrophin-3 | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| neurotrophin-4 | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| ciliary neurotrophic factor (CNTF) | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| leukemia inhibitory factor (LIF) | leukemia, neurodegenerative and |
| | neuromuscular disease including |
| | Huntington's, Alzheimer's, Parkinson's |
| | disease, multiple sclerosis, motor neuron |
| | disease, amyotrophic lateral sclerosis, ataxia, |
| | muscular dystrophy, spinal muscular atrophy, |
| | stroke, spinal cord injury, Charcot-Marie- |
| | Tooth disease, progressive supranuclear palsy |
| cholinergic differentiation factor | leukemia, neurodegenerative and |
| | neuromuscular disease including |
| | Huntington's, Alzheimer's, Parkinson's |
| | disease, multiple sclerosis, motor neuron |
| | disease, amyotrophic lateral sclerosis, ataxia, |
| | muscular dystrophy, spinal muscular atrophy, |
| | stroke, spinal cord injury, Charcot-Marie- |
| | Tooth disease, progressive supranuclear palsy |
| cardiotrophin-1 | cardiovascular disease, including cardiac |
| | myopathy |
| oncostatin M ligand | chronic inflammatory disease, including |
| | arthritis |
| tumor necrosis factor (TNF) | brain cancer, viral infections, lyme disease, |
| | tropical diseases including ascariasis, Buruli |
| | ulcer, Chagas disease, dracunculiasis, |
| | hookworm infection, human African |
| | trypanosomiasis, Leishmaniasis, leprosy, |
| | lymphatic filariasis, onchocerciasis, |
| | schistosomiasis, trachoma, and trichuriasis. |
| Neu differentiation factor | brain cancer |
| heregulin | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| acetylcholine receptor-inducing activity | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| glial growth factors (GGFs) | Schizophrenia, neurodegenerative and |
| | neuromuscular disease including |
| | Huntington's, Alzheimer's, Parkinson's |
| | disease, multiple sclerosis, motor neuron |
| | disease, amyotrophic lateral sclerosis, ataxia, |
| | muscular dystrophy, spinal muscular atrophy, |
| | stroke, spinal cord injury, Charcot-Marie- |
| | Tooth disease, progressive supranuclear palsy |
| glial cell line derived neurotrophic factor | neurodegenerative and neuromuscular disease |
| (GDNF) | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| artemin | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| neurturin | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| persephin | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| osteogenic protein-1 (OP-1) | spondylolisthesis, posterolateral arthrodesis, |
| | osteoporosis, spinal fusion, bone healing |
| bone morphogenetic proteins (BMPs) | bone healing, spinal fusion, osteoporosis, |
| growth differentiation factors | Parkinson's Disease |
| ephrin | immune disorders |
| epidermal growth factor (EGF) | necrotizing enterocolitis, Zollinger-Ellison |
| | syndrome, gastrointestinal ulceration and |
| | congenital microvillus atrophy. |
| insulin-like growth factors (IGF) | type 1 and type 2 diabetes mellitus, |
| | severe insulin resistance syndromes. Crohn's |
| | disease, juvenile chronic arthritis, or cystic |
| | fibrosis. |
| platelet-derived growth factor (PDGF) | ocular neovascularization |
| hepatocyte growth factor (HGF) | tissue repair |
| granulocyte-colony stimulating factor (G- | Neutropenia |
| CSF) | |
| serine protease inhibitors | immune disorders, auto-immune disorders, |
| | bacterial and viral immunity, cardiovascular |
| | disease, blood clotting disorders |
| protease nexin-1, | blood clotting disorders |
| hedgehog family of inducing proteins | Cancer |
| agrin | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive supranuclear |
| | palsy |
| laminin 2 | Alport syndrome, epidermolysis bullosa, |
| | neuromuscular disease including |
| | Huntington's, Alzheimer's, Parkinson's |
| | disease, multiple sclerosis, motor neuron |
| | disease, amyotrophic lateral sclerosis, ataxia, |
| | muscular dystrophy, spinal muscular atrophy, |
| | stroke, spinal cord injury, Charcot-Marie- |
| | Tooth disease, progressive supranuclear palsy |
| neuroimmunophilins | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| pigment epithelium-derived factor (PEDF) | Cancer |
| activity-dependent neuroprotective protein | schizophrenia, autism, neurodegenerative and |
| (ADNP) | neuromuscular disease including |
| | Huntington's, Alzheimer's, Parkinson's |
| | disease, multiple sclerosis, motor neuron |
| | disease, amyotrophic lateral sclerosis, ataxia, |
| | muscular dystrophy, spinal muscular atrophy, |
| | stroke, spinal cord injury, Charcot-Marie- |
| | Tooth disease, progressive supranuclear palsy |
| neuritin (activity-induced neurotrophic factor) | cerebral ischemia, depression, schizophrenia, |
| | neurodegenerative and neuromuscular disease |
| | including Huntington's, Alzheimer's, |
| | Parkinson's disease, multiple sclerosis, motor |
| | neuron disease, amyotrophic lateral sclerosis, |
| | ataxia, muscular dystrophy, spinal muscular |
| | atrophy, stroke, spinal cord injury, Charcot- |
| | Marie-Tooth disease, progressive |
| | supranuclear palsy |
| cerebral dopamine neurotrophic factor | neurodegenerative and neuromuscular disease |
| (CDNF) | including Parkinson's, Huntington's, |
| | Alzheimer's, disease, multiple sclerosis, |
| | motor neuron disease, amyotrophic lateral |
| | sclerosis, ataxia, muscular dystrophy, spinal |
| | muscular atrophy, stroke, spinal cord injury, |
| | Charcot-Marie-Tooth disease, progressive |
| | supranuclear palsy |
| mesencephalic astrocyte-derived neurotrophic | neurodegenerative and neuromuscular disease |
| factor (MANF) | including Parkinson's, Huntington's, |
| | Alzheimer's, disease, multiple sclerosis, |
| | motor neuron disease, amyotrophic lateral |
| | sclerosis, ataxia, muscular dystrophy, spinal |
| | muscular atrophy, stroke, spinal cord injury, |
| | Charcot-Marie-Tooth disease, progressive |
| | supranuclear palsy |
| Growth Hormone releasing Peptide-6 | growth hormone deficiency, wound healing |
| davunetide (derived from ADNP) | neurodegenerative and neuromuscular |
| | disease, including Parkinson's, Huntington's, |
| | Alzheimer's, disease, multiple sclerosis, |
| | motor neuron disease, amyotrophic lateral |
| | sclerosis, ataxia, muscular dystrophy, spinal |
| | muscular atrophy, stroke, spinal cord injury, |
| | Charcot-Marie-Tooth disease, progressive |
| | supranuclear palsy |
| cerebrolysin | ischemic and hemorrhagic stroke, traumatic |
| | brain injuries (TBI), dementia (vascular |
| | dementia, Alzheimer's disease) and cognitive |
| | disorders, prevention of cognitive decline |
| | after brain injuries |
| oncostatin M | chronic Inflammatory Disease, arthritis |

In specific embodiments, the disorder includes hypoprolactinemia, hypocalcemia, Prader-Willi syndrome, obesity, hypertriglyceridemia, lipodystrophy (Berardinelli-Seip syndrome, Lawrence syndrome, and Barraquer-Simons syndrome), hemophilia, Gaucher's disease, Fabry's disease, Pompe's disease, mucopolysaccharidosis, Wolman disease, hereditary angioedema, chronic neutropenia, renal anemia, thalassemia beta, hemophilia-like disease including hemophilia A and B, von Willebrand disease, factor XIII deficiency, thrombotic thrombocytopenic purpura (TTP), hemolytic-uremic syndrome (HUS), phenylketonuria, pulmonary emphysema, hypophosphatasia, cystic fibrosis, depression, pain, neurotransmission-related diseases, Parkinson's disease, ADD, anxiety, memory loss, rheumatoid arthritis (RA), cryopyrin-associated periodic syndromes (CAPS), and Alzheimer's disease, diabetes, metabolic syndrome, schizophrenia, autism, seizure-related disease, eating disorders, stress disorders, gastrointestinal disease, leukemia, cancer, cardiovascular disease, lyme disease, tropical diseases, brain cancer, necrotizing enterocolitis, Zollinger-Ellison syndrome, gastrointestinal ulceration and congenital microvillus atrophy, spondylolisthesis, posterolateral arthrodesis, osteoporosis, spinal fusion, bone healing, immune disorders, auto-immune disorders, cardiovascular disease, blood clotting disorders, Alport syndrome, epidermolysis bullosa, type 1 and type 2 diabetes mellitus, severe insulin resistance syndromes, Crohn's disease, arthritis, juvenile chronic arthritis, cystic fibrosis, ocular neovascularization, growth hormone deficiency, wound healing, tissue repair, neutropenia, neurofibroma, diabetic neuropathy, neurodegenerative and neuromuscular disease, Huntington's disease, chronic inflammatory disease, ischemic and hemorrhagic stroke, traumatic brain injuries (TBI), dementia, multiple sclerosis, motor neuron disease, amyotrophic lateral sclerosis, ataxia, muscular dystrophy, spinal muscular atrophy, stroke, spinal cord injury, Charcot-Marie-Tooth disease, and progressive supranuclear palsy. In other specific embodiments, these disorders to be treated and the polypeptide expressed by the engineered pancreatic islet organoid of the disclosure for treating the disorder are listed in the same row of Table 1.

In one specific embodiment, the organoids may comprise pancreatic beta cells comprising a recombinant expression vector, wherein the expression vector comprises a nucleic acid encoding leptin under the control of an insulin promoter. The organoids may, for example, be surgically implanted into the anterior chamber of the eye or into the brain. Transplantation of the organoid results in the production and release of leptin from pancreatic beta cells, which do not normally express leptin. This embodiment can result in the lowering of blood glucose, and insulin levels can be used to treat obesity, hypertriglyceridemia, lipodystrophy (Berardinelli-Seip syndrome, Lawrence syndrome, and Barraquer-Simons syndrome.

In other non-limiting embodiments, one or more of GLP-1, GLP-1 analogues that are resistant to degradation by DPP4, and ligands that can interact with GLP-1 receptors and other receptors (including GIP receptors and glucagon receptors), can be expressed and released from the engineered pancreatic islets organoids after transplantation, for example, transplantation into the eye or brain. Examples of GLP-1, GLP-1 analogues, ligands that can interact with GLP-1 receptors and other receptors include, but are not limited to dulaglutide, exenatide, liraglutide, lixisenatide, semaglutide, LY2944876/TT401, SAR425899, MEDI0382, HM12525A/JNJ-64565111, ZP2929/BI 456906, MK-8521, NN9277, RG7697/NNC0090-2746, DA2GIP-Oxm, [dA2]GLP-1/glucagon, Y1-dA2-I12-N17-V18-I27-G28, 29-glucagon, MAR423, and HM15211. Such embodiment can be used to treat any illness associated with GLP-1, GIP, glucagon, and/or oxymodulin including but not limited to, diabetes, obesity, metabolic syndrome, and neurologic disorders including but not limited to Parkinson's disease.

In other non-limiting examples, the pancreatic organoids can comprise beta cells comprising a recombinant expression vector, wherein the expression vector comprises a nucleic acid encoding one or more of the GLP-1, GLP-1 analogues, and/or ligands that can interact with GLP-1 receptors. In various examples of this embodiment, recombinant pancreatic organoids comprising beta cells expressing GLP-1 or GLP-1 analogues can be surgically implanted into any location including in the anterior chamber of the eye or in the brain. Surgical implantation in the brain can include implantation into a location adjacent to, either on the outside or inside, the dura mater. In various examples of this embodiment, the recombinant expression vector can comprise any suitable promoter including, but not limited to, the insulin and glucagon promoters. In this embodiment, the beta cells are engineered to secrete GLP-1 and can thus function as a positive autocrine feedback signal resulting in increased insulin secretion, from the beta cells, when blood glucose concentration is high. In one specific embodiment, the engineered pancreatic organoid comprises pancreatic beta cells, comprising a recombinant expression vector, wherein the expression vector comprises a nucleic acid encoding one or more of the GLP-1, GLP-1 analogues, and/or ligands that can interact with GLP-1 receptors under the control of the insulin promoter. In this specific embodiment, the engineered pancreatic organoids can be implanted into the brain and used to treat diabetes, obesity, metabolic syndrome and neurologic disorders, including Parkinson's disease.

Any suitable expression of the polypeptide in the subject can provide a benefit (i.e.: 10%, 15%, 20%, 25%, 30%, 35%, 40%, 45%, 50%, 55%, 60% 65%, 70%, 75%, 80%, 85%, 90%, 95%, or more, as a percentage of the expression level in a subject that does not have the disorder) in the treatment of the disorder.

### Example 1: Intraocular Genetically Engineered Pancreatic Islet Organoids as Production Sites for Blood-Born Peptides/Proteins

### Methods

Animals. Female B6.Cg-Lep^{ob}/J (ob/ob) mice and B6.BKS(D)-Lepr^{db}/J mice, which both have a C57BL/6J background, were purchased at 2 months of age from Charles River Laboratories (Wilmington, MA, USA). After delivery, the mice were allowed to adapt to the animal core facility for 1 week before the start of the experiment. All mice were group-housed on a 12/12-h dark/light cycle with free access to food (chow diet R70 from Lantmannen, Stockholm, Sweden) and water. All experiments were performed in accordance with the Karolinska Institutet's guidelines for the care and use of animals in research and were approved by the institute's Animal Ethics Committee.

Leptin treatment of ob/ob mice: Ob/ob mice were treated daily with one intraperitoneal injection of recombinant human leptin protein (1.5 μg/g bodyweight, R&D Systems, Minneapolis, USA) from the day of arrival for 5 weeks.

Expression vector construction: The mouse leptin cDNA was obtained from pCMV6.mouse leptin (OriGene, #MC208876) and subcloned into pTRE-tight (Clonetech) to create pTRE-tight.mLeptin. Next we inserted an IRES-mCherry™ sequence downstream of the mLeptin cDNA to obtain pTRE-tight.mLeptin-IRES-mCherry™. To create pENTR.TRE-tight.mLeptin-IRES-mCherry™/RIP1.DsRed2™ we exchanged in pENTR.rbGK.EGFP/RIP1.DsRed2™ (Paschen et al., FASEB J 2019) the rbGK.EGFP cassette by TRE-tight.mLeptin-IRES-mCherry™. Next we exchanged in pENTR.TRE-tight.mLeptin-IRES-mCherry™/RIP1.DsRed2™ the DsRed™ cDNA by the TetOFF-IRES-ZsGreen™ cassette obtained from pTetOFF-Dual (Green) (Clontech) thus generating pENTR.TRE-tight.mLeptin-IRES-mCherry™/RIP1.TetOFF-IRES-ZsGreen™. All constructs were verified by DNA sequence analysis. The TRE-tight.mLeptin-IRES-mCherry™/RIP1.TetOFF-IRES-ZsGreen™ cassette was transferred into the promoter-less adenovirus plasmid pAd/PL-DEST (Thermo Fisher Scientific) by the Gateway technique. The ViraPower™ Adenoviral Expression System (Thermo Fisher Scientific) was used to generate a replication-deficient adenovirus called vAd/RIP-mLeptin-OFF, which was used for transduction of cells.

Isolation of pancreatic islets: Islets were isolated from B6.BKS(D)-Lepr^{db}/J mice (db/db). Islets were prepared from mice by duct injection of collagenase (F. Hoffmann-La Roche, Basel, Switzerland) and were handpicked under a stereomicroscope MZ6 (Leica Microsystems, Wetzlar, Germany) after digestion. Thereafter islets were cultured in RPMI-1640 medium (RPMI medium), with a final concentration of 10% heat-inactivated fetal bovine serum, 2 mM glutamine, 100 U/ml penicillin, and 100 mg/ml streptomycin (RPMI medium) at 5% CO₂ and 37° C.

Islet organoid production: The islets were collected into 1.5 ml Eppendorf tubes (250 islets/tube), digested with Accutase™ (Sigma-Aldrich, St. Louis, MO, USA) for 10 min at 37° C. and centrifuged at 500 rpm. The islet cells were transduced with 4×10⁶ plaque forming units/ml of the leptin encoding adenovirus in suspension culture dishes for 1 h in RPMI medium at 5% CO₂ and 37° C. and then washed two times with an excess of RPMI medium to get rid of excess adenovirus.

2500 islet cells were seeded into each well of Nunclon Sphera™ 96U Bottom plates (Thermo Scientific, Leicestershire, UK). Islet organoids formed over a time of 5 days, after which the organoids were transferred to suspension culture dishes.

In vitro imaging of intraocular islet organoids: An inverted laser scanning confocal microscope (TCS SP8; Leica Microsystems) was used to image islet organoids as 3-dimensional stacks with a 3-μm step size. ZsGreen™ fluorescent protein was excited at 488 nm, and the fluorescence was detected at 505-535 nm, mCherry™ was excited at 561 nm, and fluorescence was detected at 580-650 nm. Backscatter signal (reflection) from the 561-nm excitation was collected at 555-565 nm.

Leptin secretion from islet organoids in vitro: Islet organoids were cultured in vitro in suspension culture dishes in RPMI medium. For glucose-stimulated leptin secretion measurements, the organoids were placed into 4-well-plate suspension dishes containing RPMI medium with 3 mM glucose for 1.5 h. After that, the organoids were first placed into new wells containing medium with 3 mM glucose for 30 min and then into new wells containing 16 mM glucose for 30 min. Then the organoids were returned into suspension culture dishes containing normal RPMI medium for continued culture. The medium from the 4-well-plate wells was collected into Eppendorf tubes, centrifuged for 1 min to pellet possible organoid fragments. The supernatants were collected into fresh tubes and kept at −20° C. until analysis.

Transplantation of islet organoids into the ACE: The islet organoids were transplanted into the ACE of 3 (first set of experiments) and 7 (second set of experiments) ob/ob recipients, using a technique previously described by Speier et al. (Speier et al., Nat Protoc 2008). Briefly, under anesthesia, organoids were transplanted into the ACE with a glass cannula after generating a puncture in the cornea with a 27-gauge needle. Great care was taken to avoid bleeding and damage to the iris. Mice were injected subcutaneous with Temgesic™ (0.1 ml/kg; RB Pharmaceuticals, Berkshire, United Kingdom) for postoperative analgesia. Each mouse of the transplanted group received 100 organoids/eye. 3 ob/ob mice (first set of experiments) and 5 ob/ob mice (second set of experiments) were used as controls and were not transplanted.

In vivo imaging of intraocular islet organoid grafts: Islet organoid grafts were imaged in vivo beginning 9 weeks after transplantation. An upright laser scanning confocal microscope (TCS SP5; Leica Microsystems), equipped with a long-distance, water-dipping objective (HXC-APO103/0.30 numerical aperture; Leica Microsystems) and a custom-built stereotaxic head holder, allowing positioning of the mouse eye containing the engrafted islets toward the objective used. Viscotear™s (Thea Nordic, Orebro, Sweden) was used as an immersion liquid between the eye and the objective, and isoflurane was used to anesthetize the mice during in vivo imaging. Grafts were imaged as 3-dimensional stacks with 3-μm step size. ZsGreen™ fluorescent protein was excited at 488 nm, and the fluorescence was detected at 505-535 nm. mCherry™ was excited at 561 nm, and fluorescence was detected at 580-650 nm. Backscatter signal (reflection) from the 561-nm excitation was collected at 555-565 nm. After imaging, the mice were allowed to recover from anesthesia. Additionally, beginning 6 weeks after transplantation, overview images of the grafts were obtained using a digital camera connected to a Leica M60 stereomicroscope while the mice were under anesthesia.

Doxycycline treatment of animals: Three animals of the transplanted group were treated with doxycycline in order to stop leptin production from the islet organoid grafts. Sterile doxycycline hydrochloride, dissolved in PBS, was administered intraperitoneally (ip) 5 times over 10 days (50 μg/kg/mouse).

Body weight and fasting and non-fasting blood glucose: Body weight and fasting blood glucose were measured after 6 h denial of food. Non-fasting blood glucose was measured at 4 μm with full access to food.

Intraperitoneal glucose tolerance test (ipGTT): To determine glucose tolerance, blood glucose levels were measured in mice that were unfed for 6 hours at basal state (0 min) and at 10, 30, 60, and 120 min after glucose injection (2 g/kg bodyweight ip, dissolved in PBS). The results were depicted as the area under the curve (AUC) of the ipGTT. Glucose concentrations were measured with the Accu-Chek™ Aviva monitoring system (F. Hoffmann-La Roche, Basel, Switzerland).

Plasma and aqueous humor samples: Blood samples were taken from non-fasted animals and collected into Microvette CB300 EDTA/PK100 tubes (Sarstedt, Nurnbrecht, Germany), centrifuged to gain blood plasma, and preserved at −20° C. until use. Aqueous humor samples were obtained at the end of the experiment and kept at −20° C. until use.

Insulin and C-peptide measurements: Ultrasensitive mouse ELISA kits (Crystal Chem, Elk Grove Village, IL, USA) were used to analyze insulin and C-peptide levels in the plasma.

Leptin measurements: Leptin was measured in cell culture medium, blood plasma and aqueous humor samples using the Mouse/Rat Leptin Quantikine™ ELISA Kit (R&D Systems, Minneapolis, USA).

Tissue extraction and sectioning: Eyes were obtained and sectioned to verify and complement data obtained in vivo. Mice were anesthetized with isoflurane and sacrificed by cervical dislocation. Eyes were extracted and fixed with 4% paraformaldehyde for 1 week. Before cryopreservation, the eyes were processed with a sucrose gradient [10-30% (wt/vol) sucrose in PBS containing 0.01% (wt/vol) sodium azide and 0.02% (wt/vol) bacitracin], embedded in OCT-Compound (Tissue-Tek, Sakura Finetek, Torrance, CA, USA), frozen in dry ice, and preserved at −80° C. until use. Then, 20 μm thick cryosections of the anterior part of the eye were collected on SuperFrost™ Plus microscope slides (VWR International, Radnor, PA, USA) and kept at −20° C. until use.

Immunofluorescence in eye sections: For immunostaining, eye sections were equilibrated to room temperature, washed, blocked, and then, incubated with the primary antibodies, goat anti m-leptin (R&D Systems, Minneapolis, USA) and rabbit anti-C-peptide (Cell Signaling, Danvers, MA, USA) in the presence of 0.1% Triton X-100 and 10% serum. After washing, secondary antibodies, anti-goat Alexa633 and anti-rabbit Alexa594 respectively (Thermo Fisher Scientific) were applied, and mounting with medium containing DAPI for nuclear counterstaining (Thermo Fisher Scientific) was performed after repeated washing. Imaging was performed using a confocal laser scanning microscope (Leica TCS SP8, Leica Microsystems) with the following excitation settings: DAPI—excitation 405 nm, detection 450-470 nm; ZsGreen™ excitation 488 nm, detection 500-525 nm; mCherry™ 548 nm, detection 560-580 nm; Alexa 594 (C-peptide staining) excitation 594 nm, detection 600-620 nm; Alexa 633 (leptin staining) excitation 633 nm, detection 640-680 nm. To avoid spectral overlap imaging was performed using in-between-frames sequential imaging.

Statistics: The values are expressed as means±SEM. A 2-sided, unpaired t test was used to determine statistical significance among different treatment groups. Statistical significance was used as follows: *P<0.05, **P<0.01, ***P<0.001. Origin 2015 64-bit (OriginLab, Northampton, MA, USA) and Excel (Microsoft, Redmond, WA, USA) were used for statistical analyses.

### Results

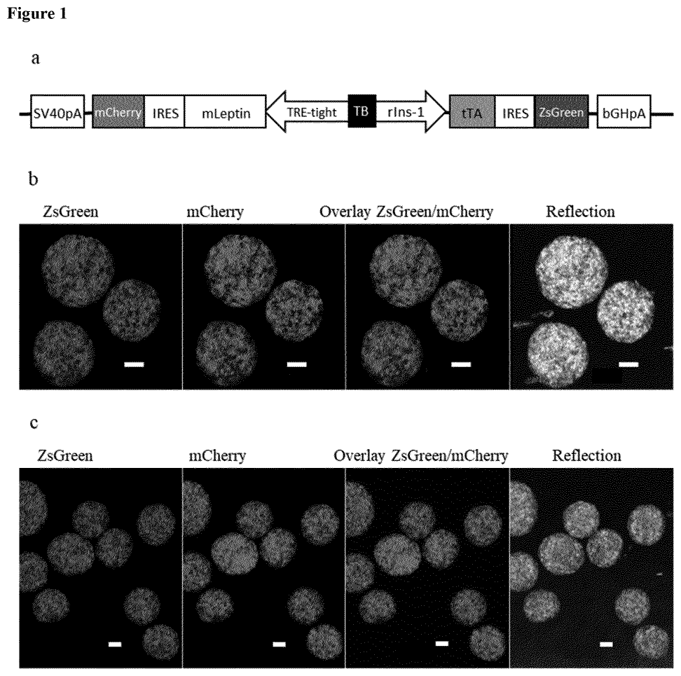

Generation of a Tet-Off-based beta-cell specific adenoviral expression construct for mouse leptin and its in vitro assessment: An adenoviral vector, vAd-RIP-leptin-OFF, that contains two expression cassettes that are positioned in opposite directions and are separated by a ‘transcription-block’ sequence to allow independent expression (FIG. 1A) was generated. The first expression cassette allows the rat insulin-1 promoter-driven expression of the synthetic transcription factor rTA (Tet-off) and the green fluorescent protein ZsGreen™ in pancreatic beta cells. The second expression cassette consists of the TRE-tight promoter driven mouse leptin-IRES-mCherry™ cassette. Binding of rTA to the TRE-tight promoter induces in the absence of doxycycline the expression of leptin and the red fluorescent protein mCherry™ in beta cells, while addition of doxycycline turns-off the expression of the two proteins. The IRES-element in the two expression cassettes allows the co-expression of rTA with ZsGreen™ and leptin with mCherry™. Hence, ‘green’ and ‘red’ serve as visual read-outs for the expression of rTA and leptin in beta cells, respectively.

For in vitro assessment, transduced organoids created from islets of B6.BKS(D)-Lepr^{db}/J mice (from the same batch as used for transplantations) were used. The detection of green and red fluorescence by laser-scanning confocal microscopy of islet organoids showed that in beta cells both expression cassettes were active (FIGS. 1B and C). The organoids secrete leptin into the culture medium, the secretion is higher at higher glucose concentrations: 7.02+/1.76 μg/organoid/h at 3 mM glucose vs. 12.94+/−2.83 μg/organoid/h at 16 mM glucose (stimulation index: 1.93+/−0.14).

Ectopic leptin production by islet organoid grafts in the ACE ameliorates the metabolic phenotype in ob/ob mice. To test whether intraocular leptin production by islet organoids affects the metabolic phenotype in ob/ob mice, the following points were considered. Because leptin deficiency after weaning leads to the rapid development of the ob/ob phenotype including strong beta-cell proliferation in both the native in situ islets as well as in islets transplanted to the ACE and leptin treatment decelerates this process (Ilegems et al., PNAS 2013), ob/ob mice were treated with daily intraperitoneal injections of leptin (1.5 μg/g bodyweight/day) immediately after their arrival from the vendor until 4 weeks after transplantation of the islet organoid grafts, i.e. after their full engraftment. The potential negative feed-back of beta-cell produced leptin was voided by using islets from leptin receptor-deficient mice for organoid generation, i.e. islets from db/db mice with a B6 genetic background (B6.BKS(D)-Lepr^{db}/J) to match the B6 background of the ob/ob recipient mice (B6.Cg-Lep^{ob}/J) Hence, we isolated islets from db/db mice, disaggregate them, transduced the islets cells with vAd-RIP-leptin-OFF and formed islet organoids by self-reassembly.

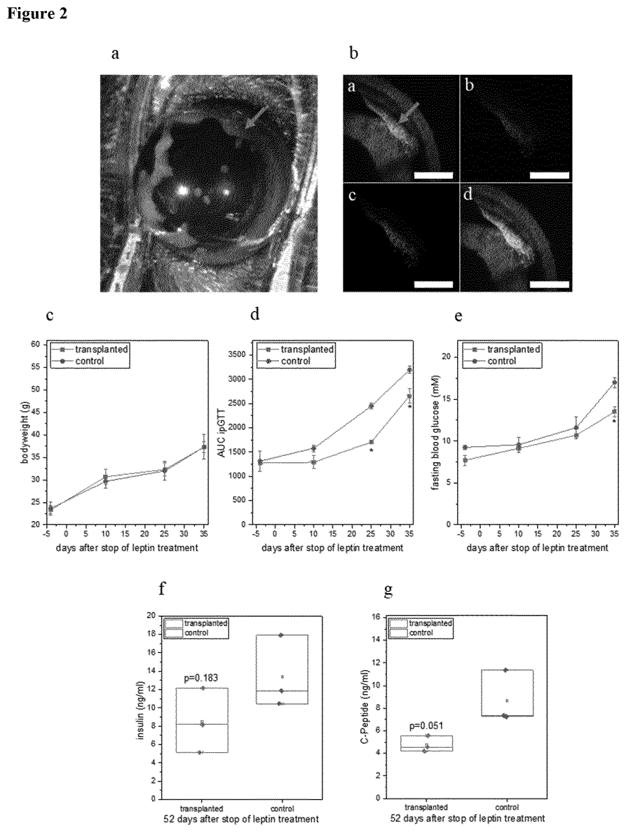

In a first set of experiment, 130 leptin-expressing db/db islet organoids were transplanted into the ACE of female ob/ob mice (n=3, see FIGS. 2A, B) that were treated with leptin from 15 d before transplantation until 28 d after transplantation and used leptin-treated female ob/ob that were not transplanted with islet organoids as a control group (n=3). Non-transduced islet organoids were not used in a control group, because earlier studies using non-transduced islets as controls for transplantation showed an excessive proliferation of these islet grafts that required termination of the experiment for ethical reasons (FIG. 2C). Inspection of islet organoids kept in vitro (FIG. 1B) as well organoids transplanted to the ACE in vivo (FIG. 2B) showed expression of both green and red fluorescent proteins, indicating that both the rTA-IRES-ZsGreen™ and Leptin-IRES-mCherry™ expression cassettes were expressed in beta cells. A difference in the bodyweight of mice between both groups (FIG. 2D) was not observed. However, a change in glucose tolerance tests (ipGTT) was observed, starting 10 d after stop of leptin treatment, which became significant 25 d after stop of leptin treatment (FIG. 2E). Moreover, in the group transplanted with leptin-producing organoids, trends towards a decrease in fasting blood glucose (FIG. 2F) was observed, which was significant at 35 d after stop of leptin treatment, towards lower plasma insulin (FIG. 2G) and insulin C-peptide (FIG. 2H) levels, both measured at the end of the experiment. Finally, leptin in the aqueous humor (11.375+/−3.211 ng/ml) was detected at the end of the experiment in the transplanted group, while no leptin was detectable in the control group. No leptin was detectable in the blood plasma from both groups.

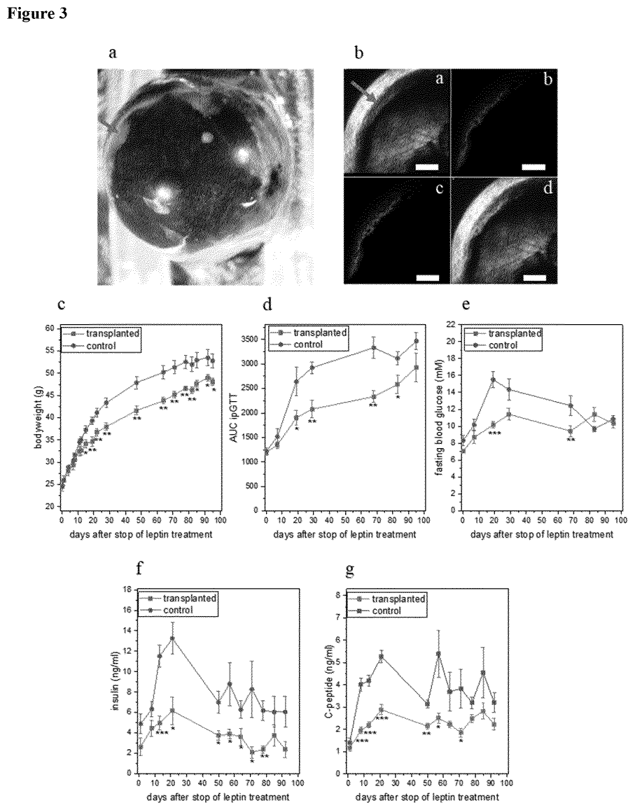

In a second set of experiments 200 leptin-expressing db/db islet organoids were transplanted into the ACE of female ob/ob mice (n=7, see FIGS. 3A, B) that were treated with leptin from 7 d before transplantation until 28 d after transplantation and used leptin-treated female ob/ob that were not transplanted with islet organoids as a control group (n=5). Inspection of islet organoids kept in vitro (FIG. 1C) as well organoids transplanted to the ACE in vivo (FIG. 3B) showed expression of both green and red fluorescent proteins, indicating that both the rTA-IRES-ZsGreen™ and Leptin-IRES-mCherry™ expression cassettes were expressed in beta cells. Importantly in this experiment, mice that were transplanted with leptin-producing organoids showed a significant difference in bodyweight from 15 d on after stop of leptin treatment (FIG. 3C). A significant change in ipGTT starting from 19 d on after stop of leptin treatment (FIG. 3D) was observed. Moreover, a decrease in fasting blood glucose (FIG. 3E, significant on days 19 and 68 after stop of leptin treatment), a significant decrease in plasma insulin (from day 14 after stop of leptin, FIG. 3F) and a significant decrease in plasma C-peptide levels (from day 8 after stop of leptin, FIG. 3G) in the group transplanted with leptin-producing organoids was observed. Finally, in that group leptin in the aqueous humor (6.05+/−2.91 ng/ml) at the end of the experiment was detected, while no leptin was detectable in the control group. The values of leptin in plasma of the transplanted group were 185.62±86.11 μg/ml between day 14 after stop of leptin treatment and the end of the experiment. No leptin was detectable in plasma from the control group.

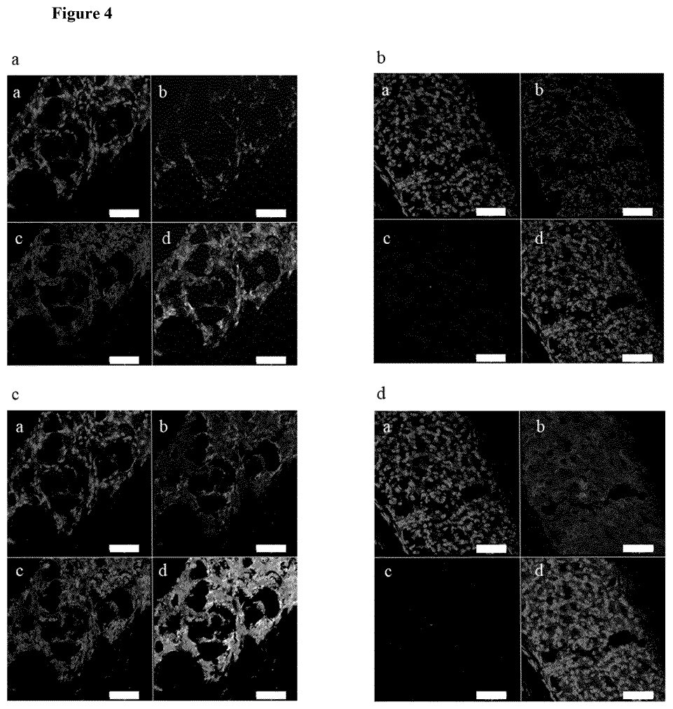

Doxycycline treatment stops ectopic leptin expression. Three animals of the transplanted group were treated with doxycycline (five intraperitoneal injections of 50 μg dox/kg/mouse over 10 days) to switch off leptin production from the transplanted organoids. Before doxycycline treatment in the grafts, the expression of both ZsGreen™ and mCherry™ was monitored. Following doxycycline treatment, in grafts of these animals no mCherry™ expression was detectable in the grafts (FIG. 4Bc) and no leptin expression could be detected by immunohistochemistry (FIG. 4Dc). In grafts of non-doxycycline treated animals mCherry™ and leptin expression were detectable (FIGS. 4A and C). More importantly also no leptin was measurable in the aqueous humor of doxycycline-treated animals (n=3).

The experimental data provide proof that genetically engineered pancreatic islet organoids can serve as production sites for proteins/peptides.

### Example 2: GLP-1 and GLP-1 Analogues, Leptin

GLP-1, GLP-1 analogues that are resistant to degradation by DPP4, and ligands that can interact with GLP-1 receptors and other receptors, including GIP receptors and glucagon receptors and other incretins can be expressed and released from the engineered pancreatic islets organoids.

We will examine the effect of engineered pancreatic islet organoids transplanted (both metabolic transplantation and reporter transplantation) to the anterior chamber of the eye, brain and the kidney capsule. These transplanted islet organoids will secrete either GLP-1 or leptin.

GLP-1 is not typically expressed in significant amounts by pancreatic cells, but is expressed by L cells in the gut. GLP-1, or synthetic ligands to GLP-1 receptor administered by injection, or orally, can have an impact on glucose control in the body, via interacting with the GLP-1 receptors in beta cells, and with central and gastric GLP-1 receptors.

We have demonstrated that the release of GLP-1, GLP-1 analogues and other ligands that can interact with the GLP-1 receptor, directly within the islet through a local autocrine feedback loop can activate the release of insulin.

The experiments include the production of adenoviruses, and pseudoislet generation and viral transduction.

### Methods

Production of Adenoviruses. Adenoviruses or adeno-associated viral vectors will be prepared using the Gateway™ cloning system (Thermo Fisher Scientific). The genetic sequence of the human GLP-1₍₇₋₃₇₎ will be placed under the CMV promoter or under the rat insulin promoter RIP1 sequence to either lead to non-cell specific expression or ensure targeted expression in insulin-producing cells, respectively.

Viral particle quantification will be performed by real-time PCR on a QuantStudio 5™ system (Thermo Fisher Scientific), using SYBR Green (Thermo Fisher Scientific) and primers specific for the adenovirus coding sequence.

Pseudoislet generation and viral transduction. For the generation of pseudoislets, islets will be dissociated into single cells by enzymatic digestion for 10 min at 37° C. using Accutase™ (Sigma-Aldrich). Cells will be counted using an automated cell counter (BRIPio-Rad) and resuspended in serum-supplemented culture medium at a density of 12 500 cells/ml. Cell suspension will be distributed on ultra-low attachment 96-well plate (Perkin Elmer) using 200 μl of cell suspension per well. Transduction will be performed by adding 1×10⁶ viral particles per 1 ml of cell suspension prior to distribution into the plate. Pseudo-islets will form and be collected after 7 days according to the manufacturer's instructions.

Planned Studies. In addition to glucose homeostasis, we will evaluate the function in the reporter islets, which show a sustained function. We will measure blood glucose levels, hemoglobin A1c, and insulin release and levels. Blood glucose levels will be shown to be lower in subjects that received transplants of islet organoids that secrete GLP-1 than subjects that did not. Insulin release will be improved in treated animals. Transplanted pancreatic islet organoids secreting GLP-1 will have use in the treatment of diabetes and obesity.

We will prepare engineered pancreatic islets organoids which express several different incretins, including, GLP-1, somatostatin and GIP, at the same time, by mixing in defined proportions and then allowing re-aggregation of islet cells of different phenotypes, transfected with different target loads. Depending on the desired time-profile associated with the target load, we will show preferential release following meals (target load in an engineered beta cell phenotype), during fasting (target load in alpha cell phenotype) and basal release.

We will transplant engineered pancreatic islets organoids into the brain to demonstrate local release of polypeptides, including GLP- and leptin. The transplant will be placed either, just outside or/and inside the dura mater in the brain.

### Results

Transplanted pancreatic islet organoids secreting GLP-1 will have use in the treatment of diabetes, obesity, metabolic syndrome as well as neurodegenerative diseases such as Parkinson's. We will show that the transplanted pancreatic islet organoids become vascularized and innervated, and secrete GLP-1 after transplantation.

Brain-transplanted pancreatic islet organoids secreting leptin will have use in the treatment of obesity, hypertriglyceridemia, lipodystrophy (Berardinelli-Seip syndrome, Lawrence syndrome, and Barraquer-Simons syndrome).

In the case of leptin secreting pancreatic islet organoids transplanted into the brain, we will perform similar measurements as in Example 1. We will show that the transplanted pancreatic islet organoids become vascularized and innervated, and secrete leptin. Further, this secretion ameliorates the metabolic phenotype in ob/ob mice, including a lowering of blood glucose, and insulin levels.

### REFERENCES

1. Åvall K, et al. (2015) Apolipoprotein CIII links islet insulin resistance to beta-cell failure in diabetes. Proc Natl Acad Sci USA 112, E2611-E2619
2. D'souza A M, et al. (2017) The glucoregulatory actions of leptin. Mol Metab 6, 1052-1065
3. Ilegems E, et al. (2013) Reporter islets in the eye reveal the plasticity of the endocrine pancreas. Proc Natl Acad sci USA 110, 20581-20586
4. Kimbrel E A and Lanza R (2020) Next—generation stem cells—ushering in a new era of cell-based therapies. Nat Rev Drug Discov 19, 463-479
5. Kulkarni R N, et al. (1997) Leptin rapidly suppresses insulin release from insulinoma cells, rat and human islets and, in vivo, in mice. J Clin Invest 100, 2729-2736
6. Leibiger I B and Berggren PO (2017) Intraocular in vivo imaging of pancreatic islet cell physiology/pathology. Mol Metab 6, 1002-1009
7. Lindström P (2007) The physiology of obese-hyperglycemic mice [ob/ob mice]. Sci World J 7, 666-685
8. Mehran A E, et al. (2012) Hyperinsulinemia drives diet-induced obesity independently of brain insulin production. Cell Metab 16, 723-737
9. Paschen M, et al. (2019) Diet-induced beta-cell insulin resistance results in reversible loss of functional beta-cell mass. FASEB J 33, 204-218
10. Speier S, et al. (2008) Noninvasive high-resolution in vivo imaging of pancreatic islet cell biology. Nat Med 14, 574-578
11. Speier S, et al. (2008) Noninvasive high-resolution in vivo imaging of cell biology in the anterior chamber of the mouse eye. Nat Protoc 3, 1278-1286
12. Van Krieken P P, et al. (2019) Translational assessment of a genetic engineering methodology to improve islet function for transplantation. EBioMedicine 45, 529-541

1. An engineered pancreatic islet organoid comprising a recombinant expression vector, wherein the recombinant expression vector comprises (a) a nucleic acid sequence encoding a polypeptide, and (b) a suitable control sequence, operatively linked to the nucleic acid sequence.

2. The engineered pancreatic islet organoid of claim 1, wherein the recombinant expression vector is present in pancreatic islet organoid cells that do not endogenously express the polypeptide.

3. The engineered pancreatic islet organoid of claim 1 wherein the polypeptide is not endogenously expressed in pancreatic islet cells

4. The engineered pancreatic islet organoid of claim 1, wherein the polypeptide is a therapeutic polypeptide, or analogues thereof.

5. The engineered pancreatic islet organoid of claim 4, wherein the therapeutic polypeptide is selected from the group consisting of polypeptide hormones and enzyme replacement proteins.

6. The engineered pancreatic islet organoid of claim 5, wherein the therapeutic polypeptide comprises a polypeptide hormone, selected from the group consisting of thyroid-stimulating hormone (TSH), prolactin, parathyroid hormone (PTH), leptin, follicle-stimulating hormone (FSH), and growth hormone (GH), or analogues thereof.

7. The engineered pancreatic islet organoid of claim 5, wherein the therapeutic polypeptide comprises a polypeptide hormone selected from the group consisting of thyrotropin-releasing hormone (TRH), renin, gastrin, vasoactive intestinal peptide (VIP), vasopressin (ADH), oxytocin (OXY), melanocyte-stimulating hormone (MSH), calcitonin, cholecystokinin (CCK), atrial natriuretic peptide (ANP), angiotensin, amylin, and adrenocorticotropic hormone (ACTH)), or analogues thereof.

8. The engineered pancreatic islet organoid of claim 5, wherein the therapeutic polypeptide comprises an enzyme replacement protein, wherein the enzyme replacement protein is selected from the group consisting of factor VII (eptacog alfa), factor VIII, factor IX, factor XIII (catridecacog), Von Willenbrand factor, taliglucerase alfa, agalsidase alfa or beta, imiglucerase, velaglucerase alfa, alglucosidase alfa, galsulfase, dornase alfa, laronidase, conestat alfa (C¹ esterase inhibitor), pegloticase alpha-1-proteinase inhibitor, asfotase alfa (Strensiq), idursulfase, elosulfase alfa valiase, selbelipase alfa, epoetin teta (Eporatio), beta (NeoRecormon) zeta (Retacrit), darbepoetin alfa (Aranesp), luspatercept (Reblozyl), filgrastim, lenograstim, and Von Willebrand factor-cleaving protease, or analogues thereof.

9. The engineered pancreatic islet organoid of claim 5, wherein the therapeutic polypeptide comprises a neurotrophic factor selected from the group consisting of enkephalin, endorphin, substance P, neurotensin, neuropeptide-Y, bombesin, brain-derived neutrophic factor (BDNF), nerve growth factor (NGF), neuotrophin-3, neurotrophin-4, ciliary neurotrophic factor (CNTF), leukemia inhibitory factor (LIF), cholinergic differentiation factor, cardiotrophin-1, oncostatin M, tumor necrosis factor (TNF), Neu differentiation factor, heregulin, acetylcholine receptor-inducing activity, glial growth factors (GGFs), glial cell line derived neurotrophic factor (GDNF), artemin, neurturin, persephin, osteogenic protein-1 (OP-1), bone morphogenetic proteins (BMPs), growth differentiation factors, ephrin, epidermal growth factor (EGF), insulin-like growth factors (IGF), platelet-derived growth factor (PDGF), hepatocyte growth factor (HGF), granulocyte-colony stimulating factor (G-CSF), serine protease inhibitors, protease nexin-1, hedgehog family of inducing proteins, agrin, laminin 2, neuroimmunophilins, pigment epithelium-derived factor (PEDF), activity-dependent neuroprotective protein (ADNP), neuritin (activity-induced neurotrophic factor), angiogenesis growth factor, cerebral dopamine neurotrophic factor (CDNF), mesencephalic astrocyte-derived neurotrophic factor (MANF), Peptide-6, davunetide (derived from ADNP), and cerebrolysin.

10. The engineered pancreatic islet organoid of claim 1, wherein the recombinant expression vector comprises a viral vector.

11. The engineered pancreatic islet organoid claim 10, wherein the viral vector is selected from the group consisting of a retroviral vector, a lentivirus vector, a murine leukemia virus (MMLV) vector, a murine stem cell virus (MSCV) vector, an adenoviral vector, a herpes simplex virus vector, a Baculovirus vector, and an adeno-associated viral vector.

12. The engineered pancreatic islet organoid of claim 1, wherein the expression vector comprises a non-viral vector.

13. The engineered pancreatic islet organoid of claim 12, wherein the non-viral vector comprises gene editing.

14. The engineered pancreatic islet organoid of claim 13, wherein the gene editing comprises the CRISPR/Cas9 system.

15. The engineered pancreatic islet organoid of claim 1, wherein the suitable control sequence is pancreatic tissue specific and is selected from the group consisting of a midkine (MK) promoter, a cyclooxygenase-2 (Cox2) M promoter, a Cox 2L promoter, a vascular endothelial growth factor (VEGF) promoter, a caveolin 1 promoter, a fms-like receptor tyrosine kinase 1 (FLT-1) promoter, a sloppy paired1 (SLP-1) promoter, a gastrin-releasing peptide (GRP) promoter, an epithelial glycoprotein 2 (EGP-2) promoter, an insulin promoter, a cytomegalovirus (CMV) promoter, a chicken β-actin (CAG) promoter, a Rous sarcoma virus (RSV), a simian virus 40 (SV40), a mammalian elongation factor 1α (EF1α) promoter, a muscle creatine kinase (MCK) promoter, a human phosphoglycerate kinase 1 (PGK1) promoter, and a tetracycline-responsive element (TRE)-tight promoter, and a glucagon promoter.

16. The engineered pancreatic islet organoid of claim 15, wherein recombinant expression vector comprises an adeno associated viral vector and the insulin promoter is RIP1 or RIP2.

17. (canceled)

18. A composition comprising the engineered pancreatic islet organoid of claim 1 and a silk matrix.

19. (canceled)

20. (canceled)

21. (canceled)

22. (canceled)

23. A method for treating a disorder, comprising implanting into a subject having a disorder the engineered pancreatic islet organoid of claim 1 in an amount effective to treat the disorder.

24-44. (canceled)

45. A process for producing an engineered pancreatic islet organoid containing islet cells, comprising the steps of:
(a) introducing a recombinant expression vector into pancreatic islet cells, wherein the recombinant expression vector comprises
(i) a nucleic acid sequence encoding a polypeptide not endogenously expressed in pancreatic islet cells, and
(ii) a suitable control sequence operatively linked to the nucleic acid sequence; and
(b) culturing the pancreatic islet cells containing the recombinant expression vector in vitro to form the engineered pancreatic islet organoid containing islet cells.

46. A process according to claim 45, wherein the introducing step (a) further comprises the steps:
(a1) preparing an aqueous mixture of the pancreatic islet cells containing the recombinant expression vector with a silk protein capable of assembling into a water-insoluble macrostructure, optionally further containing laminins;
(a2) allowing the silk protein to assemble into a water-insoluble macrostructure in the presence of the pancreatic islet cells containing the recombinant expression vector, thereby forming a 3D silk matrix for the pancreatic islet cells containing the recombinant expression vector; and
wherein the culturing step (b) involves culturing the pancreatic islet cells containing the recombinant expression vector in vitro within the silk matrix to form the engineered pancreatic islet organoid containing islet cells.

47. (canceled)


SEQUENCE LISTING (11 sequences)

SEQ ID NO: 1 - protein, 5 aa, Artificial Sequence
  Synthetic
IKVAV

SEQ ID NO: 2 - protein, 5 aa, Artificial Sequence
  Synthetic
YIGSR

SEQ ID NO: 3 - protein, 5 aa, Artificial Sequence
  Synthetic
EPDIM

SEQ ID NO: 4 - protein, 5 aa, Artificial Sequence
  Synthetic
NKDIL

SEQ ID NO: 5 - protein, 5 aa, Artificial Sequence
  Synthetic
GRKRK

SEQ ID NO: 6 - protein, 15 aa, Artificial Sequence
  Synthetic
KYGAASIKVAVSADR

SEQ ID NO: 7 - protein, 12 aa, Artificial Sequence
  Synthetic
NGEPRGDTYRAY

SEQ ID NO: 8 - protein, 11 aa, Artificial Sequence
  Synthetic
PQVTRGDVFTM

SEQ ID NO: 9 - protein, 12 aa, Artificial Sequence
  Synthetic
AVTGRGDSPASS

SEQ ID NO: 10 - protein, 8 aa, Artificial Sequence
  Synthetic
TGRGDSPA

SEQ ID NO: 11 - protein, 10 aa, Artificial Sequence
  Synthetic
CTGRGDSPAC